(19) United States
(12) Patent Application Publication
(10) Pub. No.: US 2026/0364009 A1
(43) Pub. Date: Nov. 19, 2026

(54) LIPOSOMAL POWDER FORMULATIONS OF LIPOPHILIC AND WATER-SOLUBLE SUBSTANCES

(71) Applicants: Rafael EZRA, Tel Aviv (IL); KARNAK TECHNOLOGIES, LLC, New York, NY (US)

(72) Inventor: Rafael EZRA, Tel Aviv (IL)

(73) Assignee: KARNAK TECHNOLOGIES, LLC, New York, NY (US)

(21) Appl. No.: 19/480,158

(22) PCT Filed: May 8, 2024
(86) PCT No.: PCT/IL2024/050440

§ 371 Date: Oct. 30, 2025

Related U.S. Application Data
(60) Provisional application No. 63/501,296, filed on May 10, 2023.

(51) Int. Cl. A61K 9/127; A61K 31/07; A61K 31/122; A61K 31/355; A61K 31/375; A61K 47/10; A61K 47/14; A61K 47/24; A61K 47/26

(52) U.S. Cl. CPC A61K 9/127; A61K 31/07; A61K 31/122; A61K 31/355; A61K 31/375; A61K 47/10; A61K 47/14; A61K 47/24; A61K 47/26

(57) ABSTRACT

The invention provides submicronic liposomal formulations in a dry powder form characterized in that they can incorporate various types of lipophilic and hydrophilic actives while maintaining their original structure and size under various production conditions, reconstitution mediums, varying pH, osmolarity and storage conditions.

### TECHNOLOGICAL FIELD

The invention generally pertains to the field of formulation technologies, and specifically those underlying liposomal formulations with improved properties of loading and encapsulation, extended shelf-life, and compatibility with a wide range of actives.

### BACKGROUND

In essence, successful drug delivery is in finding the right balance between maximum drug efficacy and minimum deleterious side-effects. Liposomes, due to their physicochemical and biophysical properties and inherent flexibility, are an attractive delivery system that allows to address various delivery considerations and problems of system toxicity. One advantage is that liposomes can accommodate both hydrophilic and hydrophobic substances, either in the internal aqueous core or in the lipid bilayer. Another advantage corroborated by numerous studies is that they confer stability and protection to the encapsulated actives before cellular uptake and improve their biodistribution to target sites. Nonetheless, despite the plethora of findings in this field in the past 50 years, there are some important pharmacological questions that need to be addressed to ease the translation of liposomal technology to a variety of pharmaceuticals, cosmeceuticals, and other applications.

Liposomal technology has made some steps forward, predominantly in the areas of improved drug loading using pH and ion gradients and generation of complexes of cationic liposomes and anionic nucleic acids or proteins. There are, however, some critical gaps of knowledge and operative solutions to the problems of: (1) physical and chemical instability of liposomes, especially in aqueous dispersions; (2) drug leakage and phospholipid oxidation and hydrolysis; (3) insufficient drug loading where pH and ion gradients do not apply; (3) challenges in targeting various tissues, especially via biological barriers such as skin, BBB; and (4) high costs of production, each of which hinder the application of liposomal technology and its translation to marketable liposomal products.

A general notion is that the fate, retention ability and the stability of liposomes are strongly affected by the liposomal membrane composition, rigidity, net charge, and liposome size. The liposomal membrane lipid composition is one of the critical factors. Studies have shown that manipulation of lipid membrane fluidity in favor of mechanically robust and flexible bilayers minimizes the exposure of encapsulated actives to oxidizing and hydrolyzing agents. The selection of lipids, in turn, should be commensurate with the selection of other non-active components such as phospholipids, carriers, preservatives, and further with the lipophilicity of the encapsulated active of choice. Another critical factor for liposomal stability is particle size. With smaller particles, the benefits of increased surface area and higher bioavailability usually come at the expense of reduced stability due to increased surface energy, and reduced drug loading. In addition, the methods of fabricating liposomes, such as the use of various dispersion, homogenization, mixing and lyophilization techniques, can also impact liposomal physical properties, and ultimately loading and retention capacities, and long-term stability.

Lyophilization is one of the methods to obtain improved stability and prolonged shelf-life. When applied to liposomes, it would be one of the ways to address the problem of formation of liposomal aggregates and their tendency to fuse in aqueous dispersions, and thereby resolve the problems of drug loss and drug inefficacy. In addition, a lyophilized preservable powder form would confer liposomes apparent advantages of prolonged shelf-life and can be reconstituted in biocompatible solutions prior to administration. However, one of the remaining challenges in this area is the ability to preserve the integrity of liposomal membrane during lyophilization and reconstitution processes. There are several approaches to this problem, either by the use of formulation dependent factors such as polyethylene glycol (PEG), cholesterol and cryoprotectant/ phospholipids ratios, and/or process dependent factors such as mixing, filtration and freezing conditions prior to lyophilization.

There are numerous examples of liposomal delivery systems with various types of actives. Most of them, however, fall short of providing a comprehensive solution to the problem of liposomal instability that can be applied with the same degree of success to lipophilic (hydrophobic) and water-soluble (hydrophilic) actives. Certain examples of liposomal powder formulations were reported in CN103432013B, CN105012956, CN1660119, CN106619151, CN103520006, CN1895237B.

### GENERAL DESCRIPTION

The incentive for the invention has been to find an effective and straightforward solution to one of the main problems with making successful liposomal formulations, i.e., overcoming their inherent vulnerability and instability in aqueous solutions or dispersions, and during lyophilization and reconstitution processes.

The behavior of liposomes in aqueous solutions or dispersions is characterized by physical and chemical instabilities, the first are revealed in their tendency to aggregation and fusion, the second in their tendency to degradation due to oxidation and hydrolysis. The problem of preservation of liposomal membrane integrity during lyophilization and reconstitution processes was mentioned above. Another challenge is finding the right tradeoff between liposomal drug loading and particle size, which has direct impact on drug efficacy, bioavailability, side effects, and ultimately, the applicability of liposomes.

The strength of this invention is in providing a comprehensive solution to all these problems by designing and providing proof of concept for a liposomal powder formulation with surprising advantages of accessibility, self-emulsification, prolonged stability, and conservation of its core properties in various solutions and dispersions, which in turn may ensure its exceptionally wide applicability.

One surprising property of the present formulations resides in the preservation of the original liposomal structure and nanometric or submicronic particle size through the entire length of the formulations' lifetime, starting from the preparation of the initial liposomal suspension, through lyophilization and storage of the respective powder form, and up to reconstitution of these powder formulations in aqueous solutions, suggesting that the same may apply to their behavior in the GI.

Specifically, repeated analyses of structure and size of the initial suspensions and the reconstituted powder forms showed preservation of a typical spheric phospholipid bilayer structure and a submicronic or nanometric particle size within the range of 50-500 nm and PDI/PI (polydispersity index)<0.3-0.2, suggesting similarity of particle size, and other particles' characteristics. The same particle size was substantially maintained in the initial suspensions and reconstituting solutions mimicking physiological osmolarity (250-300 mOSmoles/L) and physiological pH, and the pH of duodenum, intestine, and stomach (pH 7 to 2). The powder forms proved to be exceptionally advantageous in terms of dissolution rate, flowability, and storability under elevated temperatures. EXAMPLE 1

In addition, the method of producing these liposomal powder formulations proved to be accessible and cost effective due to the use of solid and semisolid source materials, and thus easily translatable to large scale production. Most important, these liposomal powder formulations were equally compatible with lipophilic (hydrophobic) and water-soluble (hydrophilic) actives, with the examples of Cannabidiol (CBD) in the first group and Vitamins C and B12 in the second. EXAMPLES 1-3

It was further found that the essential non-active components in these formulations are sugar(s), phospholipid(s) and alcohol(s). For specific applications, the sugars can be substituted with proteins or peptides, or combinations of sugars and proteins or peptides. Depending on lipophilicity and hydrophobicity of actives and other desired properties, the formulations can further include oil(s) or combination of oils, emulsifier(s) or surfactant(s), preservative(s), and others. Current examples of liposomal powder formulations with hydrophilic actives included a combination of simple sugars and polysaccharides, e.g., sucrose and maltodextrin, and lecithin and glycerol as choices of phospholipid and alcohol, whereas the formulations with lipophilic actives further included medium chain triglyceride (MCT). EXAMPLES 1-3

One distinguishing feature of the present formulations, most likely conferring them the specific advantages of storability and preservability of their core structural properties, resides in the proportion of the active and non-active components in the initial liposomal suspensions and the resulting powder forms. It has been presently demonstrated that the suspensions comprising about 10-50%, and specifically 20-30%, of the active and non-active components and the powders resulting from such suspensions are especially advantageous in terms of a series of demonstrable properties of preservation of core liposomal structure, particle size and improved stability in various solutions. The powders are further characterized with exceptionally advantageous flowability (no stickiness), water dispersibility (e.g., 90% dissolution rate in 30 sec) and stability equivalent to 1 year at room temperature (40 °C and 75 % RH for 90 days).

Another distinguishing feature is that the main active and non-active components are provided in solid and semisolid forms, making the process of production of these formulations exceptionally easy and translatable for large scale production.

Current efforts are focusing on producing formulations with composite actives and mixes of actives such as extracts of green tea polyphenols. EXAMPLE 4

In summary, the present invention provides a holistic formulation approach to the production of highly dependable and reconsitutable liposomal powder formulations of lipophilic as well as hydrophilic actives. The formulations of the invention, either as powders or reconstituted or dispersed forms, can be further adapted for enteral, parenteral, topical, transdermal and mucosal administrations or inhalation, depending on the type of active and its intended use. For ubiquitous applications, such as oral or sublingual, the formulations can be configured to form tablets, pills or capsules, with or without an enteric coating, and a range of other useful forms. Due to its inclusiveness and accessibility, it is anticipated that the presently proposed formulation approach can be easily incorporated into pharma, diagnostic, foods, and cosmetic industries, and in the production of various types of drugs, pharmaceuticals, nutraceuticals, food supplements, food additives, as well as biosensors, vaccines, and gene therapy products.

Ultimately, the liposomal powder formulations of the invention were meant to provide improved and safe delivery of the incorporated actives. This specific aspect is currently explored in studies evaluating the performance of the formulations by various parameters of bioavailability, bio-accessibility and biodistribution in vitro and in vivo.

### BRIEF DESCRIPTION OF THE DRAWINGS

FIG. 1 is a schematic illustration of the process of preparing the liposomal powder formulations of the invention with the example of Vit. C (ascorbic acid) as a hydrophilic active of choice.

FIGS. 2a-2b illustrate the advantageous feature of uniformity of particle size as revealed by dynamic light scattering (DLS) analysis of the reconstituted powder formulation with Vit. C in water. Pictures show a single peak with an average particle size (Z-Average) of about 200 nm, polydispersity index (PDI) of about 0.1976(<0.2 ) and an intercept of about 0.97(~1 ).

FIGS. 3a-3f illustrate the main distinguishing features of the present formulations, i.e., preservation of particle size and a typical liposomal structure after lyophilization and reconstitution, as revealed by scanning electron microscopy (SEM) of the formulations with Vit. C. Pictures show images of the initial liposomal suspensions (3a-3c) and the reconstituted powders in water (3d-3f), both exhibiting a typical liposomal structure and particles size of about 100-500 nm (arrow bars).

FIGS. 4-6 illustrate the effects of lyophilization, salinity and pH on particle size by DLS analysis, using the initial liposomal suspension and the reconstituted formulation with Vit. C.

FIG. 4 shows that before and after freeze-lyophilization the nanometric (submicronic) particle size was substantially maintained at the range of less than about 500 nm (diameter), about 300 nm, about 200 nm, or less than about 100 nm.

FIGS. 5a-5b show that the same particle size was substantially maintained after altering salinity in the initial suspension and the reconstitution solutions from 0.9% NaCl (physiological) to 1.8% NaCl.

FIGS. 6a-6c show that the same particle size was substantially maintained after altering pH in the initial suspension and the reconstitution solutions from pH=7.5 (mimicking duodenum and small intestine) to pH=2 (stomach) and then back to pH=7.

FIGS. 7a-7b illustrate that the distinctive features of preservation and uniformity of particle size were further maintained in the formulations with CBD (lipophilic active). Pictures show DLS analysis of the initial liposomal suspension and the respective reconstituted powder in water, yielding a single peak of an average particle size of about 160 nm, and PDI of about 0.155 and 0.1319 (<0.2), respectively.

FIGS. 8a-8b are SEM images of the liposomal CBD formulation as the initial aqueous suspension and the reconstituted powder, showing a typical liposomal structure of an average size of less than about 300 nm (arrows) and.

FIGS. 9a-9b are transmission electron cryomicroscopy (CryoTEM) images of the formulation with B12 (hydrophilic) as the initial liposomal suspension and the reconstituted powder, showing a characteristic liposomal structure with a nanometric (submicronic) particle size in the range of less than about 500-300 nm.

### DETAILED DESCRIPTION OF EMBODIMENTS

In the most elementary sense, the formulations of the invention can be articulated in terms of formulations comprising submicronic liposomal particles with improved ability to retain various lipophilic and hydrophilic actives. The formulations of the invention can be provided in a powder form and in the form of suspensions, solutions and or dispersions of such powder formulations.

The terms “dispersion”, “suspension” and “solution” herein are overlapping and are meant to encompass a wide range of consistencies from clear to hazy solutions, including situations in which a solute is completely or partially dissolved in a solvent, and situations in which a solute or a dispersant is present in a solvent as a homogenously or heterogeneously dispersed particulate matter of similar or various size. In the context of the invention, these terms refer herein to the initial or source suspensions for making the powder formulations of the invention and to the suspensions of the powder formulations reconstituted in various solutions, i.e., reconstituted powder formulations.

In numerous embodiments the source suspensions and/or the reconstituted suspensions of the formulations are aqueous suspensions.

Thus, in some embodiments the formulations of the invention can be articulated in terms of solid self-emulsifying water-dispersible powder formulations comprising or containing a plurality of submicronic liposomal particles comprising one or more hydrophilic or hydrophobic active components or ingredients.

In some embodiments the formulations can be articulated in terms of aqueous suspensions of submicronic liposomal particles comprising one or more hydrophilic or hydrophobic active components or ingredients.

In some embodiments the formulations can be articulated in terms of aqueous suspensions of submicronic liposomal particles for use in a method of preparation of the powder formulations as above.

One of the characteristic features of all these formulations resides in the specific combination of the non-active components, the prerequisites of which are at least one sugar and/or at least one protein or peptide, at least one phospholipid and at least one alcohol.

Another distinguishing feature is the proportion of the active and the non-active components in the source suspensions and the respective powder formulations.

In some embodiments the source suspensions can comprise about 10-50% wt active and non-active ingredients per total weight of the suspension, or more specifically can comprise about 10-50%, 10-40%, 10-30% wt of the total weight of the suspension, or about 20-50%, 20-40%, 20-30% wt of the total weight of the suspension (w/w).

In other words, in some embodiments the active and non-active ingredients can constitute about 10-50% wt of the total weight of the source suspension.

In some embodiments the active and non-active ingredients can constitute about 20-30% wt of the total weight of the suspension.

In some embodiments the active and non-active components and/or ingredients can be solid or semisolid at room temperature.

As a result, the proportion of the active and non-active ingredients in the respective powder formulations can be by 2-, 3-, 4-, 5-, 6-, 7- and 8-times higher.

In some embodiments the powder formulations can comprise about 10-99% wt active and non-active ingredients per the total weight of the powder formulation, or more specifically can comprise about 10-99%, 20-99%, 30-99%, 40-99%, 50-99%, 60-99%, 70-99%, 80-99% and 90-99% of the total weight of the formulation.

In other words, in some embodiments the active and non-active ingredients can constitute about 10-99% of the total weight of the powder formulation.

In some embodiments the active and non-active ingredients can constitute about 20-99% of the total weight of the formulation.

In some embodiments the active and non-active ingredients can constitute about 30-99% of the total weight of the formulation.

In some embodiments the active and non-active ingredients can constitute about 40-99% of the total weight of the formulation.

In some embodiments the active and non-active ingredients can constitute about 50-99% of the total weight of the formulation.

In some embodiments the active and non-active ingredients can constitute about 60-99% of the total weight of the formulation.

In some embodiments the active and non-active ingredients can constitute about 70-99% of the total weight of the formulation.

In some embodiments the active and non-active ingredients can constitute about 80-99% of the total weight of the formulation.

In numerous embodiments the powder formulations can further comprise up to about 10% water or more specifically, up to about 1%, 2%, 3%, 4%, 5%, 6%, 7%, 8%, 9% or 10% water.

Yet another distinguishing feature of the present formulations resides in the structure and size of the liposomal particles and the preservation of this structure and size under various conditions.

Liposomes are essentially a subtype of micrometric particles characterized by spherical or multilayered spherical vesicle structure produced by the self-assembly of diacyl-chain phospholipids (lipid bilayer) in aqueous solutions. Liposomes are classified into four categories based on size and number of bilayers: small unilamellar vesicles (SUV), large unilamellar vesicles (LUV), multilamellar vesicle (MLV), and multivesicular vesicles (MVV).

The term “liposomal structure” encompasses herein all these classes.

The term “particle size” refers herein to the particles' average diameter as determined by known in the art methods, such as dynamic light scattering (DLS) for example. This term is often used herein in conjunction with “particle size distribution” to convey a degree of homogeneity of particle size in a given population of particles, as reflected by the calculations of polydispersity index (PDI).

These terms predominantly refer herein to the structure and size of liposomal particles in the source suspensions of the formulations and the reconstituted formulations of the invention, and specifically in the aqueous suspensions and formulations reconstituted in aqueous solutions.

In numerous embodiments the size of liposomal particles is substantially maintained at a submicronic or a nanometric range of about 10-900 nm, or more specifically, 10-900 nm, 10-800 nm, 10-700 nm, 10-600 nm, 10-500 nm, 10-400 nm, 10-300 nm, 10-200 nm, 10-100 nm and 10-50 nm, or 50-900 nm, 50-800 nm, 50-700 nm, 50-600 nm, 50-500 nm, 50-400 nm, 50-300 nm, 50-200 nm and 50-100 nm.

The terms “substantially maintained”, “substantially similar” or “substantially the same”, which herein interchangeable, are meant to convey herein similarity or preservation of particle size or particle size distribution across various conditions. These terms encompass herein ranges of up to about 5%, 10%, 15%, 20%, 25%, 30%, 35%, 40%, 45 and 50% deviation of particle size.

In some embodiments the size of liposomal particles in the source suspensions and the reconstituted formulations can be less than about 500 nm, or less than about 400, 300, 200, 100 or 50 nm.

In some embodiments the size of liposomal particles in the source suspensions and the reconstituted formulations can be in the range of about 10-500 nm.

In some embodiments the size of liposomal particles in the source suspensions and the reconstituted formulations can be in the range of about 50-300 nm.

In other words, the size of liposomal particles in the source suspensions and the reconstituted powder forms of the invention is substantially maintained at a specific submicronic or nanometric range or under a specific nanometric threshold.

More surprisingly, the size of liposomal particles in the source suspensions and the reconstituted powder forms is substantially similar, accounting for a deviation of up to about 5%, 10%, 15%, 20%, 25%, 30%, 35%, 40%, 45 and 50% in particle size.

Furthermore, these surprising properties of present formulations, i.e., preservation of the core liposomal structure and particle size and uniformity of the particle size (or particle size distribution), are further revealed in their resilience to fluctuating conditions of salinity and pH in the aqueous source suspensions and reconstitution solutions, which suggests that the same can be expected in the gut and body fluids.

In some embodiments the liposomal particles' size can be substantially the same in the aqueous suspensions and reconstitution solutions with an osmolarity in the range of about 200-800 mOsmol/Kg water, or about 200-400, 400-600, 600-800 mOsmol per Kg or Liter water, or about 200-300, 300-400, 400-500, 500-600, 600-700, 700-800 mOsmol per Kg or Liter water.

For example, the normal range of plasma osmolarity is about 275-290 mOsmol per Kg water, and the osmolarity of urine is 500-850 mOsmol per Kg.

Similarly, in some embodiments the liposomal particles' size the liposomal particles' size can be substantially the same in the aqueous suspensions and reconstitution solutions with a pH ranging between about 1-10, or a pH of about 1-10, 1-9, 1-8, 1-7, 1-6, 1-5, 1-4, 1-3 and 1-2 or about 1-10, 2-10, 3-10, 4-10, 5-10, 6-10, 7-10, 8-10 and 9-10.

For example, the normal range of pH in the stomach is pH 1.5-3.5, in the duodenum and small intestine is pH 6-7.4 and in plasma is pH 7.35-7.45.

Thus, in some embodiments the liposomal particles' size can remain substantially the same in the aqueous suspensions and reconstitution solutions with physiological osmolarity and/or physiological pH.

In some embodiments the liposomal particles' size can be substantially the same in the aqueous suspensions and reconstitution solutions having pH of about 7±1.

The feature of preservation of or uniformity particle size is usually accompanied by the feature of uniformity of particle size distribution as reflected by PDI parameters. PDI values in the range of 0.1-0.3 usually indicate well-dispersed samples lacking detectable aggregates, a PDI of less than 0.2 is preferable.

In some embodiments the suspensions and the reconstituted formulations can have a PDI in the range of about 0.1-0.5, 0.1-0.4, 0.1-0.3 or 0.1-0.2, or less than about 0.5, 0.4, 0.3 or less than about 0.2.

Taken together, in some embodiments the suspensions and the reconstituted formulations can have a particle size in the range of about 10-500 nm and a PDI of less than about 0.3.

In some embodiments the suspensions and the reconstituted formulations can have a particle size in the range between 50-300 nm with a PDI of less than about 0.2, considered to be highly homogenous and uniform liposomal dispersions or solutions.

The totality of the afore-mentioned features are further responsible for exceptional advantages of flowability (no stickiness), water dispersibility and stability, which are characteristic of the present powder formulations.

In some embodiments the powder formulations can have dissolution rate of up to about 50%, 60%, 70%, 80%, 90% and 100% during 5, 10, 15, 20, 25, 30, 35, 40, 45, 50, 55, 60 sec as determined by an in vitro assay.

In some embodiments the powder formulations can have dissolution rate of up to about 90% during 30 sec as determined by an in vitro assay.

In some embodiments the powder formulations can remain stable at room temperature during at least 1, 2, 3, 4, 5, 6, 7, 8, 9, 10, 11, 12 months, and 2, 3, 4, 5, 6, years, or more.

In some embodiments the powder formulations can remain stable at 40 °C and 75 % RH for up to 90 days, or more.

In terms of non-active core components of the present suspensions and powder formulations, in numerous embodiments they comprise one or more sugars, which can be substituted or partially substituted with proteins or peptides, and further one or more phospholipids and alcohols, which can be natural, synthetic, and modified natural materials.

These groups further encompass herein the entire scope of materials from these groups that are permitted under the regulations of FDA or EMA or DSHEA or identified as GRASS for the purpose of medicinal, cosmetic or food applications.

In numerous embodiments the sugars can be monosaccharides, disaccharides, trisaccharides, oligosaccharides and polysaccharides, or combinations thereof.

In some embodiments the polysaccharides can be selected from the groups of natural or synthetic polysaccharides, agars, carrageenans, gellans, gelatins, guar gums, alginates and xanthan gum.

In some embodiments one or more sugars can be replaced with proteins or peptides or combinations thereof, or combinations thereof with sugars.

The term “peptide” generally refers to less than 50 amino acids, and in most cases 10-20 amino acids.

In some embodiments the phospholipids can be glycerophospholipids or sphingophospholipids, or any combination thereof.

In some embodiments the phospholipids can be selected from the groups of phosphatidylcholine (PC), phosphatidylethanolamine (PE), phosphatidylglycerol (PG), phosphatidylinositol (PI), or phosphatidylserine (PS), sphingomyelin (SM) or any combination thereof.

In some embodiments at least one of the phospholipids can be lecithin.

In some embodiments the alcohols can be primary, secondary and tertiary alcohols.

In some embodiments the alcohols can be unsaturated alcohols.

In some embodiments the alcohols can be simple alcohols.

In some embodiments at least one of the alcohols can be glycerol.

In some embodiments the non-active ingredients can be at least one of:
sucrose and/or maltodextrin as a choice of sugars,
lecithin as a choice of phospholipid, and/or
glycerol as a choice of alcohol.

In some embodiments, and especially those pertaining to lipophilic actives, the formulations can further comprise one or more oils, which can be natural, synthetic, or artificially modified natural oils.

In some embodiments one or more oils can be natural, synthetic or modified medium chain triglyceride (an MCT).

Term “MCT” unusually refers to a triglyceride with 2-3 fatty acids having an aliphatic tail of 6-12 carbon atoms, i.e., medium chain fatty acids (MCFA).

In some embodiments the formulation can further comprise one or more emulsifiers, which can be natural, synthetic or modified natural emulsifier.

The term “emulsifier” encompasses herein a wide range of substances, the common feature of which is the ability to stabilize an emulsion by reducing the oil-water interface tension. This term often overlaps herein with the term “surfactant”, which encompasses similar molecules classified according to charge as anionic, non-ionic, cationic and amphoteric surfactants.

In some embodiments an emulsifier or a surfactant can be a saponin, or a triterpene glycoside.

In terms of active components, one of the important advantages of the present formulations is in their wide-ranging applicability to different types of actives, and actives with different hydrophilic and hydrophobic properties.

The terms “hydrophilic” and “hydrophobic” encompass herein all relevant active materials as defined by negative and positive log P, respectively. These terms further encompass herein the entire scope of active materials from these groups that are permitted under the regulations of FDA or EMA or DSHEA or identified as GRASS for the purpose of medicinal, cosmetic or food applications.

In some embodiments the suspensions and the powder formulations of the invention can incorporate hydrophilic or hydrophobic actives pertaining to food and supplement industries.

In some embodiments they can comprise active ingredients selected from the groups of nutraceuticals, vitamins, dietary or food supplements, nutrients, superfoods, natural extracts of plant or animal sources, probiotic microorganisms or probiotic supporting substances or combinations or mixtures thereof.

In some embodiments the suspensions and the powder formulations of the invention can incorporate hydrophilic or hydrophobic actives pertaining to cosmetic industry.

In some embodiments they can comprise actives defined as cosmeceuticals, including cosmetically beneficial active ingredients and oils.

Still in other embodiments the suspensions and the powder formulations of the invention can incorporate hydrophilic or hydrophobic actives pertaining to pharma and diagnostic industries.

In some embodiments they can comprise actives selected from pharmaceuticals, including therapeutic or diagnostic active ingredients.

In some embodiments they can comprise actives selected from peptides, proteins, enzymes, ion channels, inhibitors, oligonucleotides, and single or double stranded DNAs or RNAs.

In some embodiments they can comprise one or more actives selected from Vitamin C, Vitamin E, Vitamin K, Vitamin A or a Vitamin from the B group. Formulations of this type have been presently exemplified.

In terms of loading and encapsulation capacities of the incorporated actives, in some embodiments in the suspensions and the powder formulations of the invention can have a loading capacity in the range of about 10-80% wt relative to the dry liposomal weight, and specifically in the range of at least about 10-80%, 10-70%, 20-60%, 30-50% wt relative to the dry liposomal weight, or in the range of at least about 10-20%, 20-30%, 30-40%, 40-50%, 50-60%, 60-70% and 70-80% wt per the dry liposomal weight (w/w).

In numerous embodiments the formulations can have a loading capacity in the range of at least about 10-80% wt relative to the weight of the liposomal water component, and specifically in the range of at least about 10-80%, 10-70%, 20-60%, 30-50% wt relative to the weight of the liposomal water component, or in the range of at least about 10-20%, 20-30%, 30-40%, 40-50%, 50-60%, 60-70% and 70-80% wt per the weight of the liposomal water component (w/w).

In numerous embodiments the formulations can have an encapsulation capacity in the range of at least about 20-80%, and specifically in the range of at least about 20-70%, 30-70%, 40-70%, 50-70%, 60-70% and 70-80%, or in the range of at least 20%, 30%, 40%, 50%, 60%, 70% or at least 80%.

In some embodiments the formulations can have an encapsulation capacity of at least about 20%, 30%, 40%, 50%, 60%, 70%, 80%, 90% and above 90%.

In some embodiments the formulations can have an encapsulation capacity in the range of 40-80%, or in the range of 40-80%, 40-70%, 40-60% and 40-50%, or 50-80%, 60-80% and 70-80%.

Certain attention should be given to the production method, in numerous embodiments the method for producing the present compositions involves steps of:
(i) mixing the active ingredient(s) with non-active ingredients in water to produce an aqueous mix,
(ii) homogenizing the aqueous mix to produce an aqueous liposomal suspension,
(iii) drying the liposomal suspension.

In some embodiments, and especially those pertaining to hydrophobic actives, the method can further include:
(iv) adding at least one oil to the aqueous mix in step (i).

Further, in numerous embodiments the homogenizing step (ii) can involve low shear homogenization and/or high-pressure homogenization.

In numerous embodiments the drying step (iii) can involve at least one of lyophilization, freeze-lyophilization or spray drying.

In some embodiments the active and the active and the non-active ingredients can be solid or semisolid at room temperature.

In some embodiments the active and the non-active ingredients constitute about 10-50% wt of the initial aqueous mix (i) or the aqueous liposomal suspension (ii).

In some embodiments, especially those pertaining to hydrophobic actives, the non-active ingredients can further comprise one or more oils.

In summary, the present formulation approach can serve as a basis for a wide range of compositions or base materials pertaining to food, cosmetics, pharma and diagnostics industries, including pharmaceutical and cosmetic compositions, food supplements and additives, and compositions comprising diagnostic probes.

In some embodiments the compositions can comprise one or more powder formulations as above, or aqueous dispersions or solutions thereof.

In some embodiments the compositions can further comprise additional carriers or excipients, depending on their intended use.

In some embodiments the compositions can be adapted for enteral, parenteral, topical, transdermal and/or mucosal administration or inhalation.

In some embodiments the compositions can be adapted for oral and/or sublingual administrations.

In some embodiments the compositions can be in the form of tablets, pills, or capsules, with or without an enteric coating.

Ultimately, the formulations and compositions of the invention are meant to improve delivery of the encapsulated actives, or more specifically, improving delivery, bio-accessibility and/or bioavailability of the comprised hydrophilic or hydrophobic actives.

In some embodiments the formulations and compositions of the invention can be used for treating specific disorders and conditions in humans and mammals in general.

In some embodiments the formulations and compositions of the invention can be used for improving certain cosmetic conditions, including humidification, moisturization, smoothing, wrinkling, aging and other conditions of the skin.

In some embodiments the formulations and compositions of the invention can be used for diagnosing certain disorders and conditions.

In some embodiments the formulations and compositions of the invention can be use to produce foods, supplements, food additives and other materials.

The terms “about” or “approximately” are meant to encompass herein a deviation of up to ±15% from the specified values or ranges, and more specifically up to a deviation of about ±1%, ±2%, ±3%, ±4%, ±5%, ±6%, ±7%, ±8%, ±9%, ±10%, ±11%, ±12%, ±13%, ±14% or ±15% therefrom.

### EXAMPLES

The figures and examples serve herein only illustrative purposes and are not intended to be limiting, as the scope of invention is decided by the scope of the claims.

### Example 1: Formulations With Hydrophilic Actives—the Example of Vitamin C

### 1.1 Background

Vit. C intake in humans has been related to reduced oxidative stress and improved physiological functions. The oral intake of Vit. C is less effective than intravenous, mainly due to its limited oral bioavailability. It was further shown that Vit. C in the form of liposomal liquid emulsions may have increased oral bioavailability compared to free Vit. C. However, liquid liposomal formulations usually suffer from physical and chemical instabilities, resulting in reduced shelf-life and loss of active during long-term storage of these preparations.

### 1.2 Objective

The main goal was to achieve a water-dispersible self-emulsifying liposomal powder formulation with Vit. C, which maintains its original structure under a wide range of conditions, and upon reconstitution of the powder form in aqueous solution.

### 1.3 Preparation Process

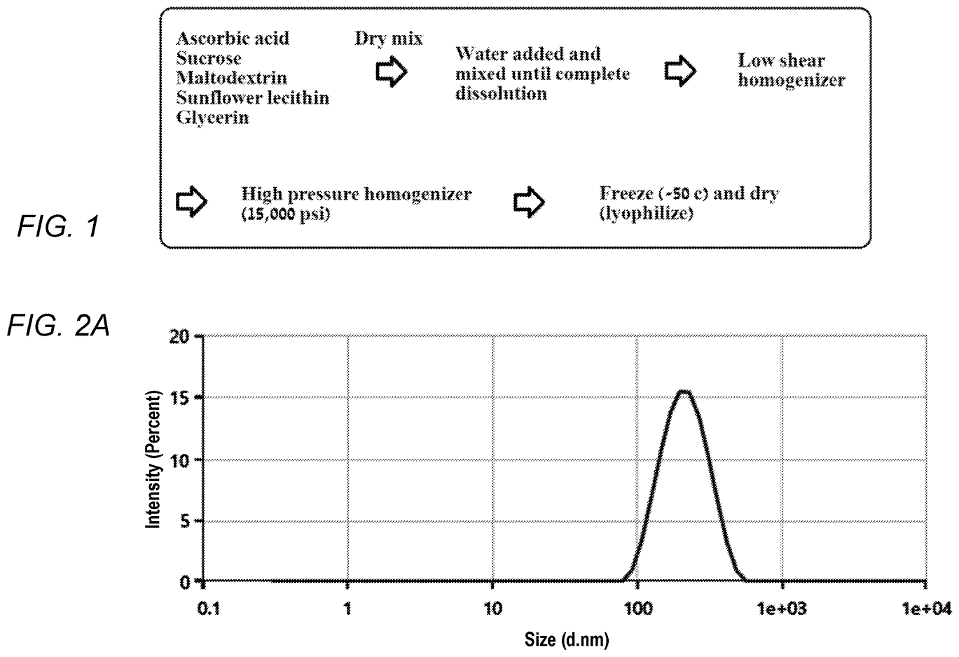

Prototype liposomal powder formulations were prepared from Ascorbic acid (Vit. C), Sucrose, Maltodextrin, Sunflower Lecithin, as solid ingredients, and Glycerin, and other additives. The initial liposomal suspensions were prepared by mixing and homogenizing the ingredients in water by low-shear and high-pressure homogenization to achieve a specific range of particle size. The powder formulations were produced by freeze-drying/lyophilization of the suspensions. Schematical illustration of the production process is shown in FIG. 1.

The reconstituted powder formulations were tested by dynamic light scattering (DLS), scanning electron microscopy (SEM) and other methods for a range of physical and chemical properties, including preservation of typical liposomal structure, particle size, dissolution or dispersibility, as well as the effect of lyophilization, salinity and pH on these properties, i.e., stability. The effect of different proportions of solid ingredients and water in the initial suspension were also tested.

### 1.4 DLS and SME Studies of an Exemplar Powder Formulation

An initial liposomal suspension was produced in two steps:
(i) by mixing Vit. C, Lecithin, Sucrose, Glycerin in certain proportions, and only partial amounts of Maltodextrin (20% total) and water (70% total), and by homogenizing the mix,
(ii) adding the rest of Maltodextrin and water, (80% and 30% respectively) immediately after homogenization and before freeze-drying/lyophilization.

This process of preparation proved to be reproducible and effective in terms of uniformity of particle size and minimization of leftover material. As a general observation, lower proportions of solid ingredients in the initial suspension (i.e., a higher proportion of water) produced more flowable and less “sticky” powders.

An example recipe of formulations with Vit. C is provided below.
Solid or semisolid components (% wt)
   Ascorbic acid—70%
   Maltodextrin—11.75%
   Sucrose—11.75%
   Sunflower lecithin (powder)—5%
   Glycerin—1%
   Ammonium Glycyrrhizate—0.5%, and
Water in the amount of about ×3 times of the solid components, i.e., 25% solid components in total suspension (% wt).

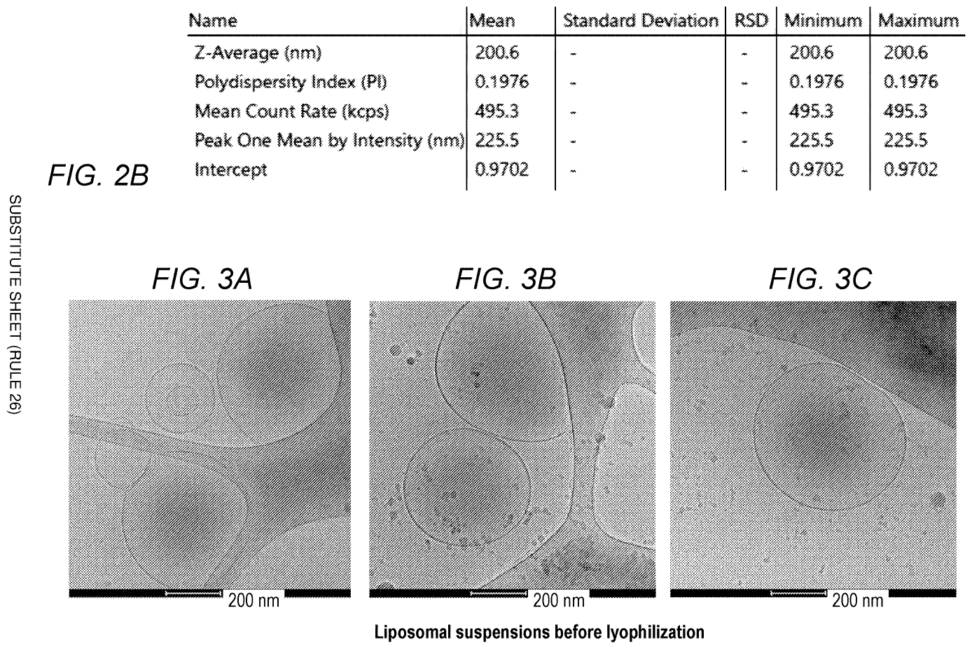

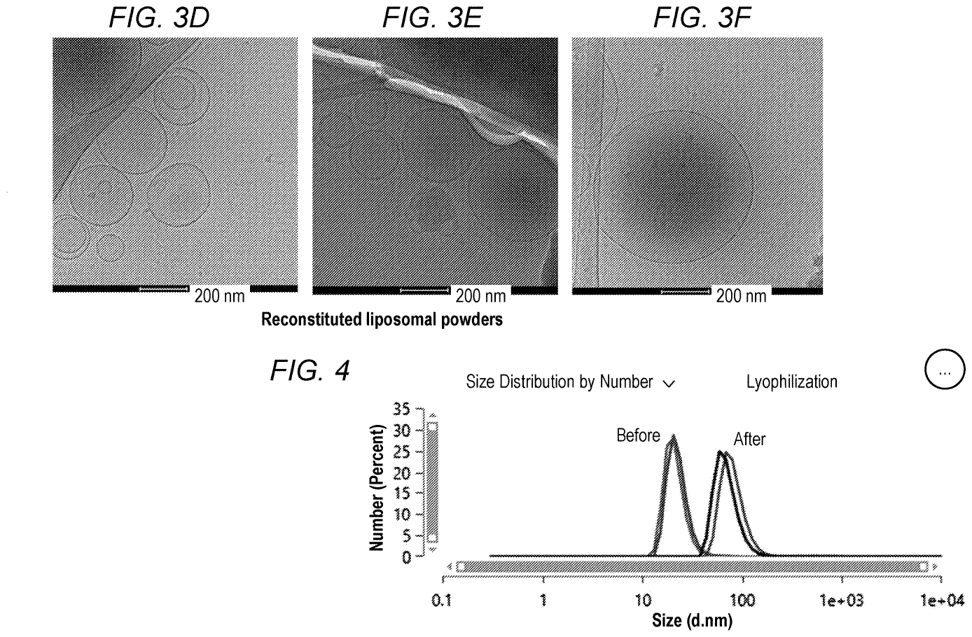

Certain physical characteristics of the reconstituted formulation as above are shown in FIGS. 2a-2b (DLS analysis) and FIGS. 3a-3f (SEM imaging). Specifically, the formulation of the invention exhibited surprising preservation and uniformity of particles in a submicronic size range (FIGS. 2a-2b), with average particle size (Z-Average) of about 200 nm, PDI of about 0.1976(<0.2 ) and an intercept of about 0.97 intercept (~1 high reliability). In addition, the initial liposomal suspension (before lyophilization) and the reconstituted powder further exhibited surprising preservation of a typical liposomal structure with particle size within the same submicronic range (FIGS. 3a-3f).

The same formulation was further tested for pH and dissolution time (1 gr powder in 20 mL water) and flowability and stability of the powder under prolonged storage. These properties are listed below:
average pH in water 3.5,
dissolution time (90% dissolution) in water 30 sec,
stability and flowability of the powder at 40 °C and 75 % RH for 90 days (equivalent to 1 year at RT), with no change in Vit. C content, dissolution time and pH upon reconstitution of the powder in water (up to 5% error).

### 1.5 the Effects of Lyophilization, Salinity and pH

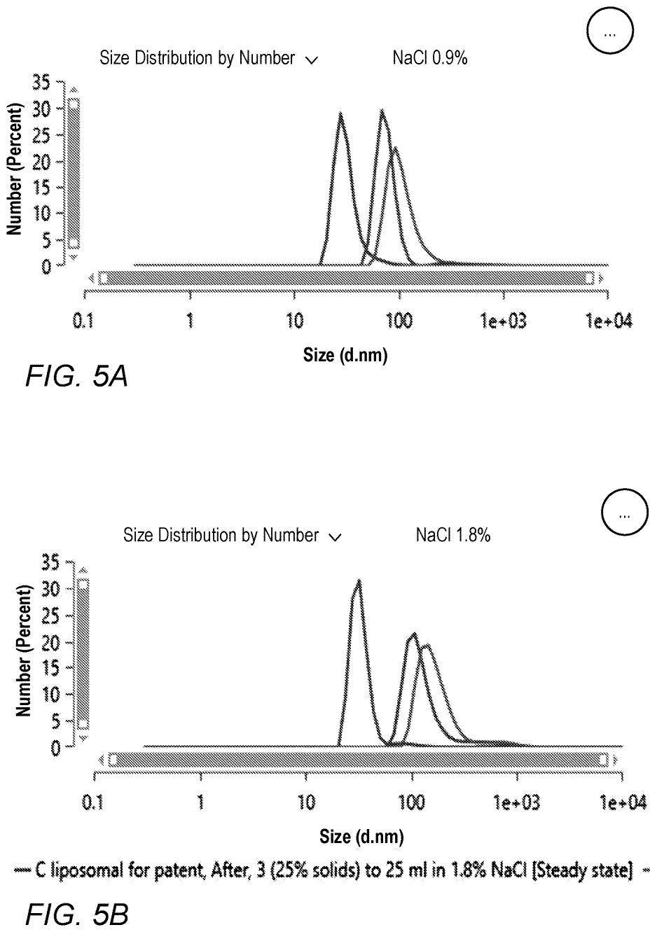

The reconstituted powder formulation (25% solid components) was further tested for the effects of lyophilization, and salinity and pH on particle size (DLS analysis), comparing the initial liposomal suspension (1 mL in 25 mL water) and the reconstituted powder (1 g in 20 mL water). The results are shown in FIGS. 4-6.

Specifically, the results indicated that freeze-drying/lyophilization had no significant effect on particle size (FIG. 4), with particle size substantially maintained at an average of less than about 500 nm (diameter), less than about 300 nm, less than about 200 nm or even less than about 100 nm, before and after lyophilization.

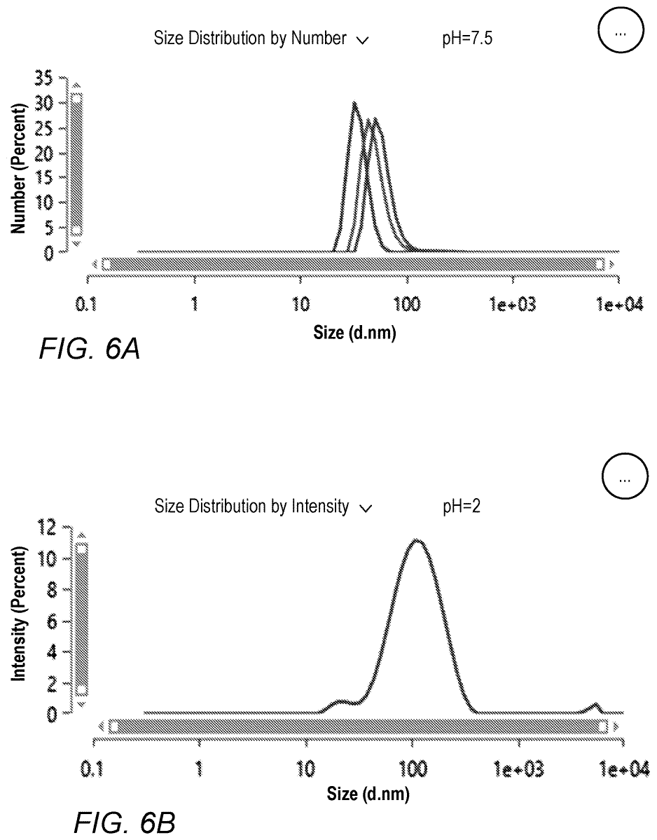

Increasing salinity from 0.9% NaCl to 1.8% NaCl had certain effect on particle size (FIGS. 5a-5b), with a tendency to form larger particles and hazy solutions in a higher salinity. Nonetheless, even in a higher salinity, the particle size was maintained at a submicronic range, and in the physiological salinity (0.9% NaCl), the particle size was preserved at an average of less than about 500 nm, less than about 300 nm, and less than about 200 nm.

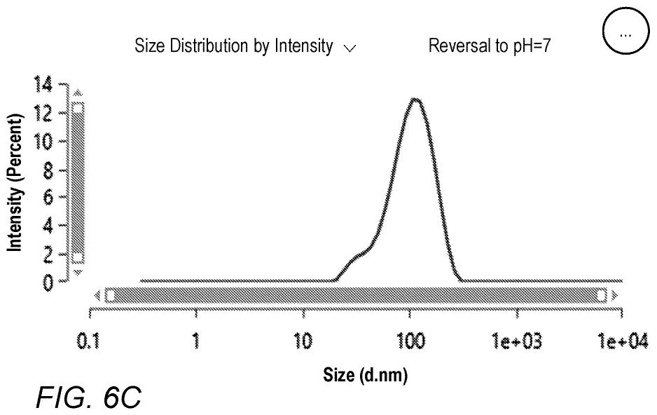

The effect of pH on particle size was insignificant (FIGS. 6a-6c), maintaining the particle size at an average of less than about 500 nm, less than about 300 nm, and less than about 200 nm in solutions with pH=7.5 (duodenum and small intestine), pH=2 (stomach) and upon reversal to pH=7.

The integrity of liposomal structure was examined by comparing the residual filtered material (centrifugal filter 100K mw cutoff, 6000 RPM for 30 min) of the liposomal suspension and the reconstituted powder (1:3 ratio in water). The results indicated that approximately the same range of residual matter remained on the filter, 45% suspension and 61% powder (% w/w), suggesting preservation of particle size.

### 1.6 Conclusions

Overall, these studies suggested that the liposomal powder formulation of the invention (about 25% wt solid components) was surprisingly successful in terms of flowability, dissolution rate and stability of the powder form, a further, in ability to preserve particle size and typical liposomal structure in the reconstituted form under a range of tested conditions.

### Example 2: Formulations With Lipophilic Actives—the Example of Cannabidiol

### 2.1 Objective

The objective was to find whether the above formulation approach can be applicable to lipophilic actives, with the example of Cannabidiol (CBD) as a highly lipophilic molecule.

### 2.2 CBD Powder Formulation

The initial liposomal suspension of CBD and the respective powder formulation were prepared from CBD and the same non-active ingredients (25% wt solid components) and by the same process (see FIG. 1). The reconstituted powder was tested for particle size, size distribution and liposomal structure using DLS and SEM analyses.

A prototype CBD formulation included: (% wt):
Solid or semi solid components (% wt)
   CBD crystals—4%
   MCT (medium chain triglyceride) oil—6%
   Sunflower lecithin (powder)—10%
   Sucrose—48%
   Maltodextrin—32%, and
Water in the amount of approx. ×3 times of the solid components, i.e., or 25% wt solid components in total suspension.

### 2.3 Particle Size and Liposomal Structure

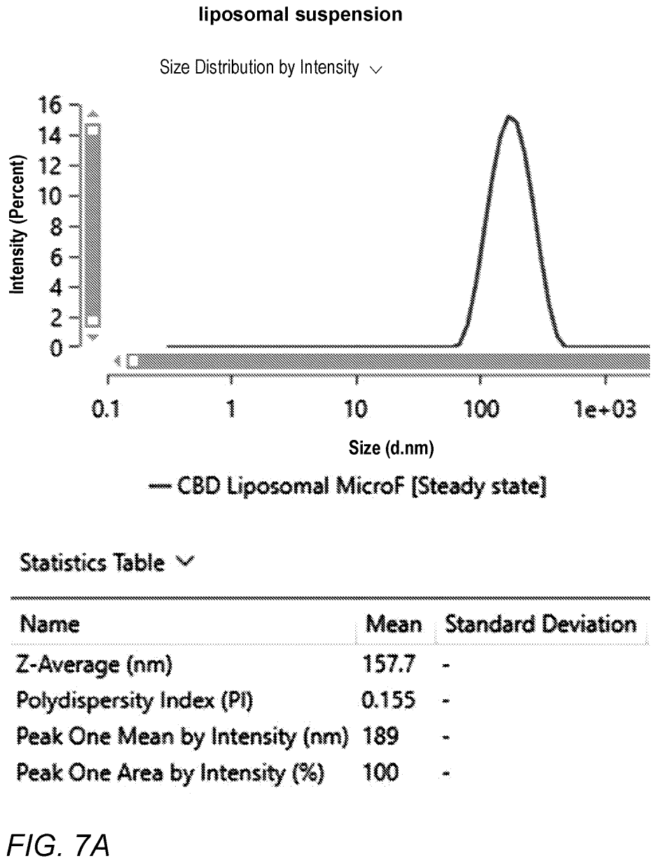

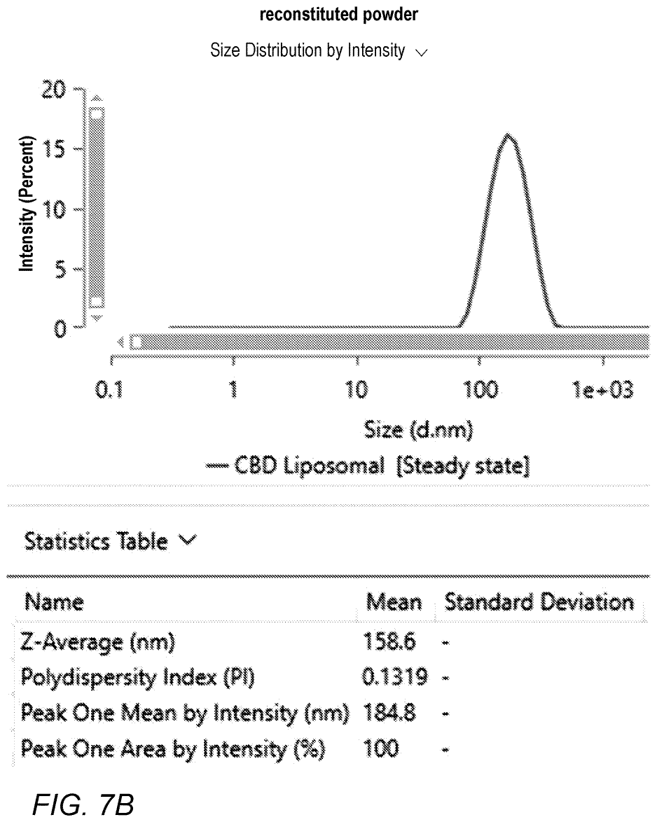

Analyses of particle size and liposomal structure in the initial suspension and the reconstituted powder are shown in FIGS. 7a-7b (DLS) and FIGS. 8a-8b (SEM). Specifically, the analysis of particle size and particle size distribution showed the same surprising property of preservation of particles (FIGS. 7 a-7b), with average size of about 200 nm (~160 nm) and PDI of about 0.155 in the liposomal suspension and 0.132 in the reconstituted powder (<0.2). The imaging analysis further supported structural preservation (FIGS. 8a-8b), showing particles of typical liposomal structure and size at an average of less than about 500 nm or less than about 300 nm.

### 2.4 Conclusions

In summary, the present formulation approach (25% solid components in the initial suspension, % w/w) proved to be equally successful for formulating lipophilic and hydrophilic actives into stable preservable self-emulsifying liposomal powder formulations with surprising properties of preservation of particle size and liposomal structure in the reconstituted form, irrespective lyophilization and storage conditions.

More recent studies suggested that the powder formulations with various types of lipophilic actives may have a loading capacity in the range of at least about 10-70% (w/w) relative to of the dry liposomal weight or in the range of at least about 10-70% (w/w) relative to the liposomal water component and an encapsulation capacity in the range of at least about 20-70%.

### Example 3: Liposomal Powder Formulation With Vitamin B12

### 3.1 Background

Vit. B12 is a water-soluble active that functions as a cofactor of many enzymes. Among its multiple functions are neuronal development, myelination and functionality of the central nervous system, and healthy red blood cell formation and DNA synthesis.

### 3.2 Objective

The importance of Vit. B12 has prompted studies using the same formulation approach to produce analogous liposomal powder formulation with Vit. B12.

3.3 Vit. B12 Powder Formulation

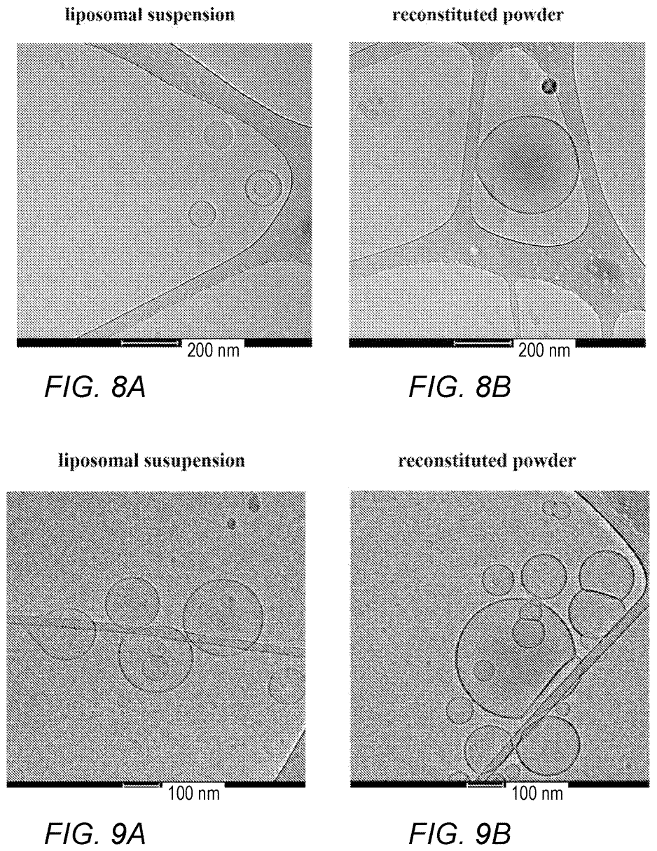

The initial liposomal suspension was prepared from the mix of solid components (25% wt relative to total weight) by the proportions and methods as above (see FIG. 1), including:
Cyanocobalamin (powder),
Sunflower lecithin (powder),
Sucrose,
Maltodextrin, and water,
   The reconstituted powder was subjected to DLS and transmission electron cryomicroscopy (CryoTEM) analyses for evaluation of particle size and liposomal structure. According to DLS (data not shown), particle size in the reconstituted powder was maintained at an average of about 200 nm (185 nm) and PDI of 0.35. The CryoTEM results are shown in FIGS. 9a-9b.

Specifically, the imaging analysis of the reconstituted form showed a characteristic liposomal structure with an aqueous core surrounded by a lipid bilayer, and average particle size in the range of less than about 500 nm, less than about 400 nm, less than bout 300 nm, or in the range of 200-500 nm (smaller liposomal particles are either superposition or fusion). In summary, the powder formulations with Vit. B12 exhibited the same surprising property of preservation of particle size and liposomal structure in the reconstituted form.

### 3.4 Conclusions

Overall, these experiments provided additional proof of concept for the general applicability of present formulation approach for incorporation of various types of lipophilic and hydrophilic actives into highly advantageous, reproducible, stable and self-emulsifying liposomal powder formulations.

### Example 4: Liposomal Powder Formulation With Complex Actives

### 4.1 Background

Polyphenols are important ingredients of green tea, constituting about 25-35% of the leaves' dry weight. They, and specifically their predominant components such as catechins, epicatechin (EC), epigallocatechin (EGC), epicatechin gallate (ECG), and epigallocatechin gallate (EGCG), have been consistently associated with anti-inflammatory, antibacterial, anti-cancer and metabolic regulatory effects, and many other medicinally beneficial actions. Polyphenols are relatively hydrophilic and are known to be extremely unstable and prone to degradation under increasing temperature, light, pH, oxygen, especially under harsh conditions in the GI tract, resulting in their relatively low oral bioavailability and bio-accessibility.

### 4.2 Objective

Additional effort was put to produce analogous liposomal powder formulations from tea polyphenols using the same formulation approach.

### 4.3 Tea Polyphenol Powder Formulation

The initial liposomal suspensions and the respective powder formulations were produced using the same proportions of active and non-active ingredients (25% wt solid components) and preparation methods (see also FIG. 1). Preliminary analyses suggested that the reconstituted powders contained liposomal particles with a typical core and lipid bilayer structure, submicronic particle size and relatively uniform size distribution. Additional studies are now conducted to evaluate loading and encapsulation capacity of these formulations, as well as stability and shelf-life, and their oral bioavailability and bio-efficacy in vitro and in vivo.

1.-80. (canceled)

81. A solid self-emulsifying water-dispersible powder formulation of submicronic liposomal particles comprising at least one hydrophilic or hydrophobic active ingredient and non-active ingredients comprising at least one phospholipid, at least one alcohol, at least one sugar and/or protein or peptide and at least one oil,
wherein the active and non-active ingredients constitute about 10-99% wt of the total weight of the formulation.

82. The formulation of claim 81, wherein the active and non-active ingredients constitute about 50-99% wt of the total weight of the formulation.

83. The formulation of claim 81, wherein the submicronic liposomal particles of the formulation reconstituted in an aqueous solution are of a size of less than about 500 nm.

84. The formulation of claim 83, wherein the size of submicronic liposomal particles remains substantially in the same range for the formulations reconstituted in the aqueous solutions with an osmolarity in the range of about 200-800 mOsm/Kg water and/or a pH in the range of about 1-10.

85. The formulation of claim 81, wherein the at least one sugar is a combination of sucrose and maltodextrin, the at least one phospholipid is lecithin, the at least one alcohol is glycerol, and the at least one oil is a medium chain triglyceride oil (an MCT).

86. The formulation of claim 81, further comprising at least one emulsifier.

87. The formulation of claim 86, wherein the at least one emulsifier is a saponin.

88. The formulation of claim 81, wherein the at least one hydrophilic or hydrophobic active ingredient is selected from at least one of the following:
(a) nutraceuticals, vitamins, dietary or food supplements, nutrients, superfoods, natural extracts of plant or animal sources, probiotic microorganisms or probiotic supporting substances, or a combination or a mixture thereof,
(b) cosmeceuticals, including cosmetically beneficial ingredients and oils,
(c) pharmaceuticals, including therapeutic and diagnostic active ingredients,
(d) a peptide, a protein, an enzyme, an ion channel, an inhibitor, an oligonucleotide, a single or a double stranded DNA or RNA,
(e) Vitamin C, Vitamin E, Vitamin K, Vitamin A or at least one Vitamin from the B group.

89. An aqueous suspension of submicronic liposomal particles comprising at least one hydrophilic or hydrophobic active ingredient and non-active ingredients comprising at least one phospholipid, at least one alcohol, at least one sugar and/or protein or peptide and at least one oil,
wherein the active and the non-active ingredients are selected from solid or semisolid at room temperature.

90. The suspension of claim 89, wherein the submicronic liposomal particles are of a size of less than about 500 nm.

91. The suspension of claim 89, wherein the at least one sugar is a combination of sucrose and maltodextrin, the at least one phospholipid is lecithin, the at least one alcohol is glycerol, and the at least one oil is a medium chain triglyceride oil (an MCT).

92. The suspension of claim 89, further comprising at least one emulsifier.

93. The suspension of claim 92, wherein the at least one emulsifier is a saponin.

94. An aqueous liposomal suspension comprising the formulation of claim 81.

95. A pharmaceutical composition comprising the formulation of claim 81, and optionally further comprising a pharmaceutically acceptable carrier or excipient, the composition is adapted for enteral, parenteral, topical, transdermal, mucosal administrations, inhalation and/or oral or sublingual administration.

96. A cosmetic composition comprising the formulation of claim 81, and optionally further comprising a cosmetically acceptable carrier or excipient.

97. A food supplement or additive comprising the formulation of claim 81.

98. A diagnostic composition comprising the formulation of claim 81 and a labelling element.

99. A method for treating a disorder or a medical or a cosmetic condition in a subject, comprising administering to the subject the formulation of claim 81, optionally in the form of a suspension or a pharmaceutical or a cosmetic composition.

100. A method for diagnosing a disorder in a subject, comprising administering to the subject the formulation of claim 81, optionally in the form of a suspension or a diagnostic composition.
